(19) United States
(12) Patent Application Publication
(10) Pub. No.: US 2002/0193374 A1
(43) Pub. Date: Dec. 19, 2002

(54) Reagents and methods for the diagnosis of CMV dissemination

(75) Inventors: Thomas J. Schall, Palo Alto, CA (US); Brian E. McMaster, Mountain View, CA (US); Daniel J. Dairaghi, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, San Carlos, CA

(21) Appl. No.: 09/944,051

(22) Filed: Aug. 30, 2001

Related U.S. Application Data
(60) Provisional application No. 60/229,191, filed on Aug. 30, 2000.

(51) Int. Cl. A61K 51/00; A61K 31/535; A61K 31/445; A61K 31/40; A61K 31/495

(52) U.S. Cl. 514/237.8; 424/1.11; 514/252.12; 514/317; 514/428

(57) ABSTRACT

Methods are provided for detecting the spread of cytomegalovirus in a host infected with CMV, by administering to the host a detectable and labeled amount of a non-endogenous compound which binds to US28 or a US28 fragment. Typically, the methods use a labeled form of IBZM.

### CROSS-REFERENCES TO RELATED APPLICATIONS

This application claims the benefit of U.S. Provisional Patent Application Serial No. 60/229,191, filed Aug. 30, 2000, the disclosure of which is incorporated herein by reference.

### STATEMENT AS TO RIGHTS TO INVENTIONS MADE UNDER FEDERALLY SPONSORED RESEARCH AND DEVELOPMENT

Not applicable

### BACKGROUND OF THE INVENTION

Cytomegalovirus (CMV) is an important human pathogen and a major opportunist which emerges to cause disease in the immuno-compromised such as AIDS patients, neonates, and individuals who have been given immunosuppressive drugs as part of a transplantation regimen. In these individuals, the consequences of CMV in acute or re-emerging infections can be dire, including retinitis, encephalitis, and pneumocystis, among other pathologies. Furthermore, in immuno-competent hosts, CMV establishes a persistent lifelong infection through which it has been linked to a variety of inflammatory conditions including coronary artery occlusion following heart transplant and arthrectomy and restenosis following angioplasty. CMV interacts with leukocytes during acute infection of the host as well as during lifelong latency. As such, leukocytes are important players in CMV-induced disease and have been implicated in the acute phase of infection as vehicles for dissemination of virus and as sites of residence during lifelong latency.

### SUMMARY OF THE INVENTION

In one aspect, the present invention provides methods for detecting the spread of cytomegalovirus in a host infected with CMV, by administering to the host a detectable and labeled amount of a non-endogenous compound which binds to US28 or a US28 fragment. Typically, the methods use a labeled form of a compound of the formula:

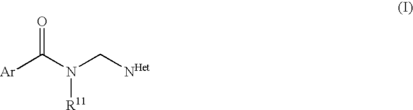

or a pharmaceutically acceptable salt thereof; wherein Ar represents a substituted aryl group; R¹¹ represents H or (C₁-C₄)alkyl; and N^{Het} is a substituted or unsubstituted 4-, 5-, 6-, or 7-membered nitrogen heterocycle.

Preferred embodiments within this group are those compounds having the formula:

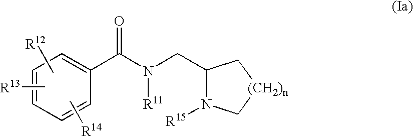

or a pharmaceutically acceptable salt thereof; wherein the subscript n is an integer of from 1 to 3; R¹¹ and R¹⁵ are independently selected from H and (C₁-C₄)alkyl; R¹², R¹³ and R¹⁴ are each members independently selected from H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; with the proviso that at least one of R¹², R¹³ and R¹⁴ is other than H.

In certain preferred embodiments within this group, n is 1, R¹¹ is H, R¹⁵ is (C₁-C₄)alkyl; and R¹², R¹³ and R¹⁴ are each other than H. In the most preferred embodiments, the compound is a labeled form of S(−)-3-Iodo-2-hydroxy-6-methoxy-N[(1-ethyl-2-pyrrolidinyl)methyl]-benzamide. A preferred labeled form is the [¹²³I]-labeled form.

In another aspect, the present invention provides methods for blocking CMV dissemination in a host by administering to the host an effective amount of a compound which blocks the binding of a chemokine to US28. Preferably, the compound is a compound represented by the formulae above. In this group of embodiments, the compound is preferably unlabeled.

### BRIEF DESCRIPTION OF THE DRAWINGS

FIG. 1 illustrates the specific displacement of chemokine (fractalkine) binding to the US28 chemokine receptor by IBZM.

FIG. 2 illustrates the Ca⁺² flux profile between IBZM and a chemokine ligand (fractalkine) for US28.

FIG. 3 illustrates the reversibility of IBZM binding to US28. In this figure, IBZM is pre-incubated with US28 expressing cells (at concentrations of 0-10 μg/mL) and removed by competition with fractalkine.

### DETAILED DESCRIPTION OF THE INVENTION

Abbreviations and Definitions

Abbreviations: CMV, cytomegalovirus; S(−)-IBZM, S(−)-3-Iodo-2-hydroxy-6-methoxy-N[(1 -ethyl-2-pyrrolidinyl)methyl]-benzamide.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. When used alone, the term “alkyl” refers to unsubstituted versions of the groups noted above. Groups provided as “substituted” are described in detail below.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl or heterocyclyl include 1 -(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien -2-yl, tetrahydrothien-3-yl, 1 -piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “(C₁-C₄)haloalkyl” is mean to include trifluoromethyl, 2,2,2-trifluoroethyl, 4-chlorobutyl, 3-bromopropyl, and the like.

The term “acyl” is used in its conventional sense and refers to an organic radical derived from an organic acid by the removal of the hydroxyl group. Examples of “acyl” groups include acetyl, propionyl, butanoyl, hexanoyl, isobutyryl, octanoyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) when indicated as “substituted” can include a variety of substituents which provide a stable moiety. Preferred substituents for each type of radical are provided below.

Substituents for the alkyl and heteroalkyl radicals (including those groups often referred to as alkoxy, alkenyl, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH′, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1 -pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Substituents for the aryl and heteroaryl groups are varied and are selected from: -halogen, —OR′, —OC(O)R′, —NR′R″, —SR′, —R′, —CN, —NO₂, —CO₂R′, —CONR′R″, C(O)R′, —OC(O)NR′R″, —NR″C(O)R′, —NR′—C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula —T—C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula —A—(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

General

CMV harbors in its genome an open reading frame (ORF), designated US28, which encodes a protein that acts as a functional receptor for certain human and viral chemokines. Upon infection of a cell by CMV, US28 is expressed on the surface of the infected cell and becomes capable of responding to chemokines in the environment. Because the virus on its own is inherently non-motile, and because chemokines and their receptors encoded by human cells are known to regulate the migration of leukocytes and other cells through the body, CMV US28 is thought to be encoded by the virus to facilitate the dissemination of CMV through the body during and after infection. Therefore, agents which block the binding of chemokines to US28 should prove useful in inhibiting viral dissemination during acute or re-emerging CMV infection.

CMV US28 has been shown to bind a number of human, murine, and virus-encoded CC chemokines in a variety of assay formats. In addition, the CX3C chemokine, Fractalkine, binds with a very high affinity (K_{I}˜50 pM) to US28. Fractalkine is expressed on certain endothelial cell surfaces and on populations of dendritic cells (DC), and may thus define a portal through which CMV infected cells go from the circulation to the tissue space, as well as find residence in the DC.

Since the US28 receptor is expressed on cytomegalovirus infected cells, and also in view of its ability to bind multiple chemokines, a small molecule which specifically binds to this receptor would have significant use as an agent to diagnose the spread of CMV, and also as an anti-CMV agent.

CMV US28 chemokine receptor is expressed on the surface of cells after infection by CMV. The receptor binds a number of chemokines and triggers viral dissemination. Accordingly, US28 (or fragments having chemokine binding activity) can be used to screen for inhibitors of chemokine binding to this receptor (see Co-pending Application Ser. No. 60/229,365, Attorney Docket No. 019934-002500US, filed Aug. 30, 2000). Additionally, compounds which bind to US28 are useful for following the dissemination of the virus in a host. We have now discovered that S(−)-3-Iodo-2-hydroxy-6-methoxy-N[(1-ethyl-2-pyrrolidinyl)methyl]benzamide (S(−)-IBZM or IBZM, from the RBI division of Sigma-Aldrich) is an effective inhibitor of the binding of native chemokine ligands (such as fractalkine and eotaxin, among others), to the chemokine receptor encoded by the US28 open reading frame of human cytomegalovirus (CMV). Moreover, this compound was found to bind specifically to US28 among all chemokine receptors tested. Historically IBZM has been known to bind to D2 dopamine receptors in humans and other species. However, the compound has not been associated with any methods for the detection, diagnosis and imaging, or treatment of CMV. The chemical structure of IBZM includes an accessible iodide moiety suitable for substitution with the radiolabeled tracer ¹²³Iodine. [¹²³I]-IBZM has been used clinically in humans and other species for imaging of the distribution of D2 dopamine receptors by SPECT or PET scanning technologies. As a result of IZBM's specific chemokine receptor binding and its ready availability in a labeled form, the compound has particular utility for in vivo detection, diagnosis, and imaging of CMV infection. Unlabeled forms of IBZM and related derivatives also have utility for treatment of CMV dissemination by blocking chemokine binding to US28 on cell surfaces, an event which triggers viral dissemination.

Description of the Embodiments

A. Methods for Detecting, Diagnosing or Imaging CMV Infection in a Host.

In one aspect, the present invention provides methods for diagnosing CMV in a host having CMV, the methods comprising:

(a) administering to the host an image-generating amount of a compound having the formula:

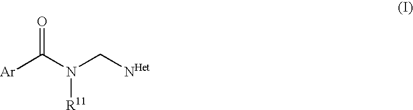

or a pharmaceutically acceptable salt thereof; wherein Ar represents a substituted aryl group; R¹¹ represents H or (C₁-C₄)alkyl; and N^{Het} is a substituted or unsubstituted 4-, 5-, 6-, or 7-membered nitrogen heterocycle; and

(b) detecting sites at which the compound binds to US28 on cell surfaces present in the host.

In one group of preferred embodiments, Ar is a substituted phenyl group. In another group of preferred embodiments, Ar is a substituted phenyl group and N^{Het} is a substituted or unsubstituted pyrrolidinyl, substituted or unsubstituted piperidinyl, substituted or unsubstituted piperazinyl, substituted or unsubstituted piperidyl or a substituted or unsubstituted morpholinyl.

More preferably, the compound has the formula:

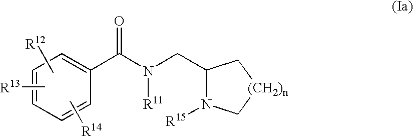

or a pharmaceutically acceptable salt thereof; wherein the subscript n is an integer of from 1 to 3; R¹¹ and R¹⁵ are independently selected from H and substituted or unsubstituted (C₁-C₄)alkyl; R¹², R¹³ and R¹⁴ are each members independently selected from H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; with the proviso that at least one of R¹², R¹³ and R¹⁴ is other than H.

In one group of embodiments, n is one; R¹¹ is H; R¹², R¹³ and R¹⁴ are each independently selected from H, hydroxy, halogen, (C₁-C₄)alkyl and (C₁-C₄)alkoxy; and R¹⁵ is (C₁-C₄)alkyl.

A variety of labeled forms of the compounds described herein are available. For example, compounds of formula I or formula Ia in which the aryl group has a halogen substituent can be prepared using a suitable isotope of the halogen atom. Additionally, the labeled atom can be readily introduced in the penultimate synthesis step. For example, benzoic acid can be radioiodinated using conventional methods, then coupled to a suitable aminomethyl(heterocycle) to form the target compound useful for imaging. Alternatively, R¹¹ or R¹⁵ can be a haloalkyl group which is incorporated into the structure in the final synthesis step.

The compounds of the invention therefore provide improved methods for imaging the CMV in a subject using PET and SPECT. The methods entail administering to a subject (which can be human or animal, for experimental and/or diagnostic purposes) an image-generating amount of a compound of the invention, labeled with the appropriate isotope and then measuring the distribution of the compound by PET if ¹⁸F or another positron emitter is employed, or SPECT if ¹²³I or another gamma emitter is employed. An image-generating amount is that amount which is at least able to provide an image in a PET or SPECT scanner, taking into account the scanner's detection sensitivity and noise level, the age of the isotope, the body size of the subject and route of administration, all such variables being exemplary of those known and accounted for by calculations and measurements known to those skilled in the art without resort to undue experimentation.

Accordingly, one of R¹², R¹³ or R¹⁴ is preferably a halogen which can be prepared in a PET-labeled, SPECT-labeled or radiolabeled form. Particularly preferred halogen labels are ¹⁸F, ⁷⁵Br, ¹²³I and ¹²⁵I. In the most preferred embodiments, one of R¹², R¹³ or R¹⁴ is iodine, and in labeled form is ¹²³I.

It is understood that compounds of the invention can be labeled with an isotope of any atom or combination of atoms in the structure. While ¹⁸F, ⁷⁵Br, ¹²³I and ¹²⁵I have been emphasized herein as being particularly useful for PET, SPECT and tracer analysis, other uses are contemplated including those flowing from physiological or pharmacological properties of stable isotope homologs and is apparent to those skilled in the art.

The compounds of formulae I and Ia can be prepared using conventional synthetic methods known to those of skill in the art. In particular, compounds of formula Ia have been described in, for example, Schmidt, et al., J. Pharm. Sci. 88(3):305-315 (1994), and in references cited therein. Other compounds are described in PCT publication WO 95/04051, WO 90/09170, U.S. Pat. No. 5,190,741 and EP 320630.

Imaging methods useful with labeled forms of IBZM other compounds of formula I and Ia have been described in, for example, Singhaniyom, et al., Brian Res. 453(1-2):393-6 (1988); Kung, et al., J. Nucl. Med. 31(5):573-9 (1990); Verhoeff, et al., Int. J. Rad. Appl. Instrum. B. 18(8):837-46 (1991); John, et al., J. Nuc. Med. 34(12):2169-75 (1993); Berding, et al., Nuklearmedizin. 33(5):194-9 (1994); Brandau, et al., J. Nucl. Med. 37(11):1865-71 (1996); Dence, et al., Nucl. Med. Biol. 24(4):333-40 (1997); Kufferle, et al., Psychopharmacology (Berl). 133(4):323-8 (1997); Zamora, et al., Life Sci. 63(18):1611-8 (1998); Dresel, et al., J. Nucl. Med. 39(7):1138-42 (1998); Tauscher, et al., Psychopharmacology (Berl). 141(2):175-81 (1999); and Klimke, et al., Psychiatry Res. 90(2):91-101 (1999).

The methods described herein are particularly useful in diagnosis of CMV in humans, however a broader application of the methods is contemplated by the present invention. For example, suitably labeled compounds can be used in other hosts which serve as models systems for new CMV treatment regimens to follow the spread of CMV in the model systems. Accordingly, the term “host” is meant to include in its broadest sense, any mammal having a CMV infection which expresses US28 on the surface of infected cells.

B. Methods of Treating CMV Infections in a Host

In another aspect, the present invention provides methods of treating CMV infection in a host, by administering to the host an effective amount of a compound which inhibits chemokine binding to US28 on the surface of CMV-infected cells. In this manner, the compound blocks CMV dissemination in the host.

In one group of embodiments, the compounds have the formula:

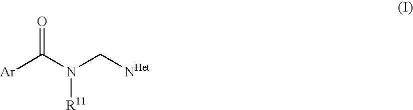

or a pharmaceutically acceptable salt thereof; wherein Ar represents a substituted aryl group; R¹¹ represents H or (C₁-C₄)alkyl; and N^{Het} is a substituted or unsubstituted 4-, 5-, 6-, or 7-membered nitrogen heterocycle.

Preferred embodiments within this group are those compounds having the formula:

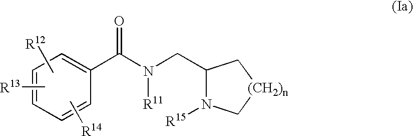

or a pharmaceutically acceptable salt thereof; wherein the subscript n is an integer of from 1 to 3; R¹¹ and R¹⁵ are independently selected from H and substituted or unsubstituted (C₁-C₄)alkyl; R¹², R¹³ and R¹⁴ are each members independently selected from H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; with the proviso that at least one of R¹², R¹³ and R¹⁴ is other than H.

In certain preferred embodiments within this group, n is 1, R¹¹ is H, R¹⁵ is (C₁-C₄)alkyl; and R¹², R¹³ and R¹⁴ are each other than H. In other preferred embodiments, n is one; R¹¹ is H; R¹², R¹³ and R¹⁴ are each independently selected from H, hydroxy, halogen, (C₁-C₄)alkyl and (C₁ -C₄)alkoxy; and R¹⁵ is (C₁-C₄)alkyl.

The methods described herein use the compounds and compositions described herein to treat disease or provide medicinal prophylaxis to individuals who possess a compromised immune system or are expected to suffer immunosuppressed conditions, such as patients prior to undergoing immunosuppressive therapy in connection with organ transplantation or anticancer chemotherapy. These methods generally involve administering to the host an effective amount of the subject compounds or pharmaceutically acceptable compositions.

The compositions and compounds of the invention and the pharmaceutically acceptable salts thereof can be administered in any effective way such as via oral, parenteral or topical routes. Generally, the compounds are administered in dosages ranging from about 2 mg up to about 2,000 mg per day, although variations will necessarily occur depending on the disease target, the patient, and the route of administration. Preferred dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day.

Therapeutic and prophylactic methods of this invention comprise the step of treating patients in a pharmaceutically acceptable manner with those compounds or compositions. Such compositions may be in the form of tablets, capsules, caplets, powders, granules, lozenges, suppositories, reconstitutable powders, or liquid preparations, such as oral or sterile parenteral solutions or suspensions. Compounds of the invention may also be administered via an intraocular implant for treating retinitis as a result of CMV infection. In particular, compounds may be embedded in a polymer based implant which will be release into the eye over an extended period of time.

Physicians will determine the dosage of the present therapeutic agents which will be most suitable. Dosages may vary with the mode of administration and the particular compound chosen. In addition, the dosage may vary with the particular patient under treatment. The dosage of the compound used in the treatment will vary, depending on viral load, the weight of the patient, the relative efficacy of the compound and the judgment of the treating physician. Such therapy may extend for several weeks or months, in an intermittent or uninterrupted manner.

C. Compositions Useful in the Treatment of CMV Infection

The present invention also provides compositions useful for preventing CMV dissemination in a host, which comprises a pharmaceutically acceptable carrier or adjuvant and an effective amount of a compound identified using the assays described herein. Preferably, the compound is a compound of formula I, more preferably, formula Ia.

Typically, the compositions contain from about 0.1% to about 99% by weight of active compound, and preferably from about 10% to about 60% by weight depending on which method of administration is employed.

A CMV dissemination-inhibiting amount is that amount of active compound required to slow the progression of viral dissemination or reduce the amount of viral dissemination from that which would otherwise occur without administration of the compound. Or, it is an amount of active compound required to slow the progression or reduce the intensity of symptoms resulting from CMV infection or elimination thereof.

CMV dissemination-inhibiting activity of compounds of the invention can be determined according to the assays described herein. The assays provide an indication of chemokine binding to US28, more typically fractalkine binding to US28. The compounds provided herein inhibit the binding of fractalkine to US28 with activity expressed as IC50 (that amount of compound that reduces fractalkine binding by 50%). The compounds provided herein will typically exhibit an IC50 of approximately 50 μg/mL or less, preferably 25 μg/mL or less, more preferably 10 μg/mL or less, and most preferably less than 1 μg/mL.

For the compositions of the invention, the proportion of each carrier, diluent or adjuvant is determined by the solubility and chemical nature of the compound and the route of administration according to standard pharmaceutical practice. In order to obtain consistency of administration, however, it is preferred that a composition of the invention is in the form of a unit dose. For example, the unit dose presentation forms for oral administration may be tablets and capsules and may contain conventional excipients such as binding agents (e.g., acacia, gelatin, sorbitol, or polyvinylpyrrolidone), fillers (e.g., lactose, sugar, maize-starch, calcium phosphate, sorbitol or glycine), tableting lubricants (e.g., magnesium stearate), disintegrants (e.g., starch, polyvinylpyrrolidone, sodium starch glycoallate or microcrystalline cellulose), or pharmaceutically acceptable wetting agents (e.g., sodium lauryl sulfate).

The compounds may be injected parenterally; this being intramuscularly, intravenously, or subcutaneously. For parenteral administration, the compound may be used in the form of sterile solutions containing other solutes, for example, sufficient saline or glucose to make the solution isotonic. The amount of active ingredient administered parenterally will be approximately 0.01 to 250 mg/kg/day, preferably about 1 to 10 mg/kg/day, more preferably about 0.5 to 30 mg/kg/day, and more most preferably about 1-20 mg/kg/day.

The compounds may be administered orally in the form of tablets, capsules, or granules containing suitable excipients such as starch, lactose, white sugar and the like. The compounds may be administered orally in the form of solutions which may contain coloring and/or flavoring agents. The compounds may also be administered sublingually in the form of tracheas or lozenges in which each active ingredient is mixed with sugar or corn syrups, flavoring agents and dyes, and then dehydrated sufficiently to make the mixture suitable for pressing into solid form. The amount of active ingredient administered orally will depend on bioavailability of the specific compound.

The solid oral compositions may be prepared by conventional methods of blending, filling, tableting, or the like. Repeated blending operations may be used to distribute the active agent throughout those compositions employing large quantities of tillers. Such operations are, of course, conventional in the art. The tablets may be coated according to methods well known in normal pharmaceutical practice, in particular with an enteric coating.

Oral liquid preparations may be in the form of emulsions, syrups, or elixirs, or may be presented as a dry product for reconstitution with water or other suitable vehicle before use. Such liquid preparations may or may not contain conventional additives. For example suspending agents, such as sorbitol, syrup, methyl cellulose, gelatin, hydroxyethylcellulose, carboxymethylcellulose, aluminum stearate gel, or hydrogenated edible fats; emulsifying agents, such as sorbitan monooleate or acaci; non-aqueous vehicles (which may include edible oils), such as almond oil, fractionated coconut oil, oily esters selected from the group consisting of glycerin, propylene glycol, ethylene glycol, and ethyl alcohol; preservatives, for instance methyl para-hydroxybenzoate, ethyl para-hydroxybenzoate, n-propyl parahydroxybenzoate, or n-butyl parahydroxybenzoate of sorbic acid; and, if desired, conventional flavoring or coloring agents.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

In another embodiment, the invention provides the subject compounds in the form of a pro-drug, which can be metabolically or chemically converted to the subject compound by the recipient host. A wide variety of pro-drug derivatives are known in the art such as those that rely on hydrolytic cleavage or oxidative activation of the prodrug.

The compositions may be advantageously combined and/or used in combination with other antiviral agents which are either therapeutic or prophylactic agents, and different from the subject compounds. The compositions may also be advantageously combined and/or used in combination with agents that treat or induce conditions often associated with the viral infections that are sensitive to the present compounds, such as anti-HIV agents or immunosuppressive agents. In many instances, administration in conjunction with the subject compositions enhances the efficacy of such agents. Exemplary antiviral agents include ganciclovir, foscarnet and cidofovir. Exemplary anti-HIV agents include indinavir, ritonavir, AZT, lamivudine and saquinavir. Exemplary immunosuppressive agents include cyclosporin and FK-506. The compositions may also be advantageously used as antiviral prophylactic treatment in combination with immunosuppressive protocols such as bone-marrow destruction (either by radiation or chemotherapy).

To further assist in understanding the present invention, the following non-limiting examples are provided.

### EXAMPLES

### Example 1

This example describes an assay for evaluating compounds which bind to US28 and inhibit the binding of chemokines. This evaluation can be beneficial in determining suitable dosage levels for either diagnostic methods or methods of treatment.

The US28 expressing cells used in most assays consist of a mouse cell line stably expressing transfected US28 cDNA under the control of a CMV promoter. These cells were cultured in IMDM-5% FBS, and harvested when the concentration was between 0.5-1.0×10⁶ cells/mL. Some assays were performed with adherent human 293 cells (US28-293 cells) or membranes. The cells were centrifuged and resuspended in assay buffer (20 mM HEPES, 140 mM NaCl, 1 mM CaCl₂, 5 mM MgCl₂, and with 0.2% bovine serum albumin) to a concentration of 5.6×10⁶ cells/mL. Using the Multi-Probe automated system, set up with 8 assay plates at a time, first 0.09 mL of cells was added to the assay plates containing the compounds. The final concentration of the compounds was 5 μg/mL each (1 μg/mL Comgenex). Then 0.09 mL of ¹²⁵I-fractalkine diluted in assay buffer (final concentration ˜2-10 fM, with ˜30,000 cpm per well) was added, the plates sealed and incubated for approximately 3 hours at 4 degrees C. on a shaker platform. The assay plates were harvested using Packard filter plates, pre-soaked in PEI solution, on the vacuum harvest apparatus. Scintillation fluid (35 μL) was added to all wells, the plates were sealed and counted in a Top Count scintillation counter. Control wells containing either diluent only (for total counts) or excess Fractalkine (1 μg/mL, for non-specific binding) were used to calculate the percent of total inhibition for each set of compounds. Further tests on individual compounds were carried out in the same manner.

### Example 2

As secondary assays for compounds that specifically inhibited the binding of radiolabled Fractalkine to US28, cytoplasmic calcium mobilization experiments were done by loading US28-293 cells with INDO-1 dye (45 min. at room temperature), washing with PBS, and resuspending into Ca²⁺ flux’ buffer (HBSS with 1% fetal bovine serum). For each test, 1×10⁶ cells were incubated at 37° C. in the cuvette of a PTI spectrometer, and the ratio of 410/490 nm emission plotted over time (typically 2-3 minutes), with compounds added at 5 seconds, followed by fractalkine at 60 seconds. A rise in intracellular Ca²⁺ is typically seen when US28-293 cells are challenged with fractalkine, an indication that the US28 receptor bound to the ligand, engaged a G-protein linked cascade which resulted in the mobilization of Ca²⁺ in the cytoplasm of the US28-bearing cells. Compounds which inhibited fractalkine binding were tested in this assay for the effects on Ca²⁺ in this system.

### Example 3

This example illustrates the effect of IBZM at inhibiting the binding of fractalkine to US28.

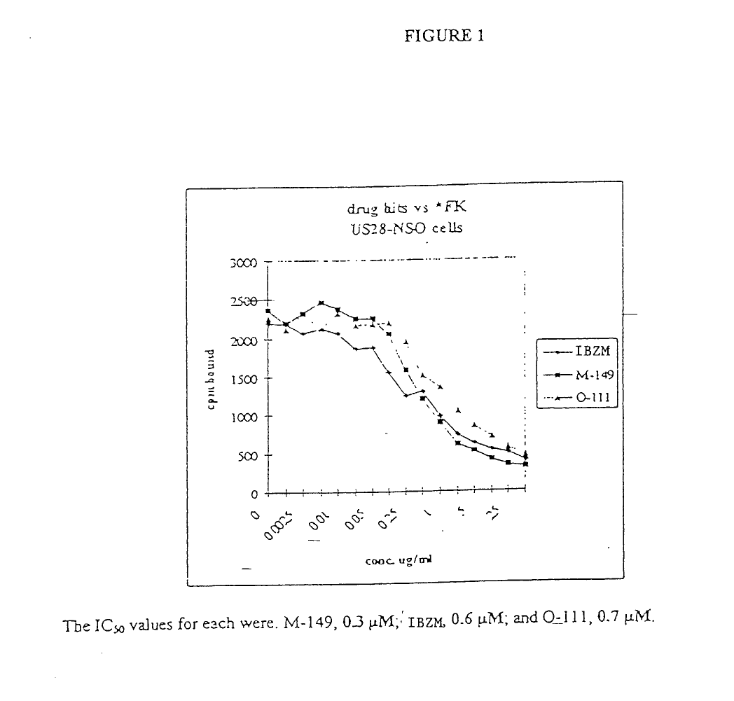

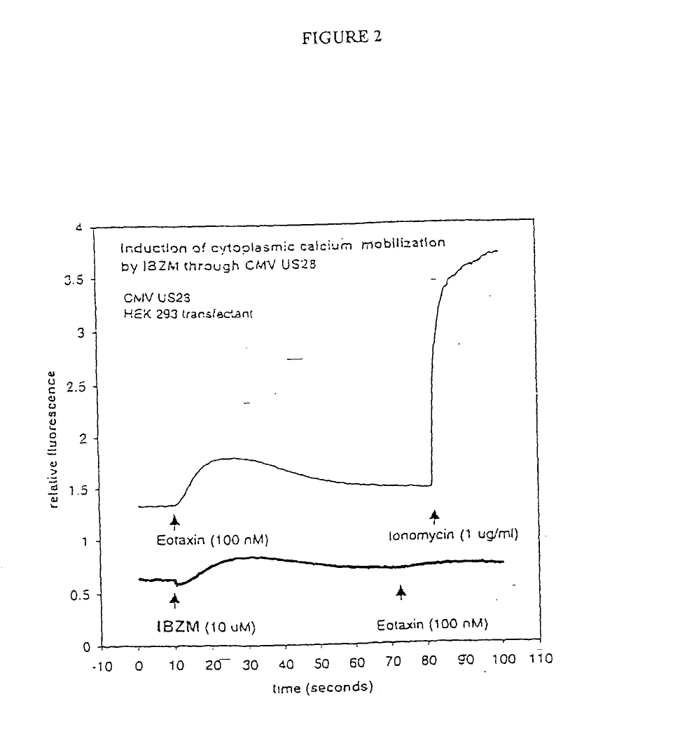

S-(−)-IBZM (from the RBI division of Sigma Chemical Co., St. Louis, Mo., USA, Catalog No. I-139) was evaluated in the assays described in Examples 1 and 2. A dose response of S(−)-IBZM against fractalkine binding on US28-NSO cells is shown in FIG. 1. The IC₅₀ value was 0.6 μM. Additionally, when the compound was tested for calcium mobilization in US28-293 cells, IBZM was found to act as a competitive agonist for the US28 receptor, mimicking the action of fractalkine in both binding and signaling (see FIG. 2).

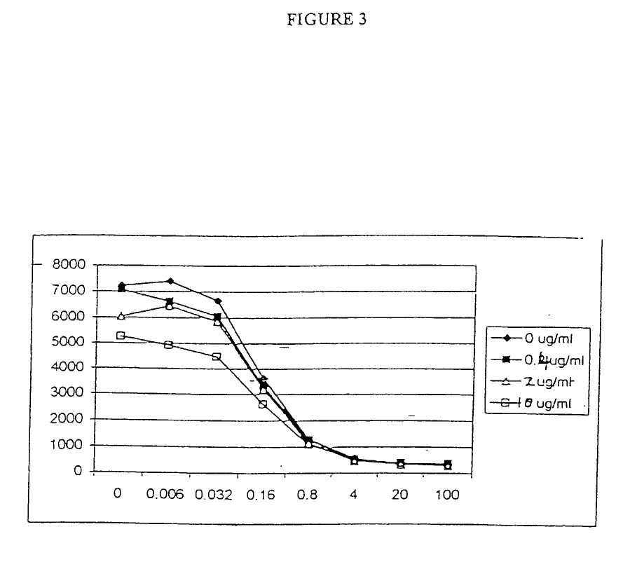

In a further study, the binding of IBZM to US28 was shown to be reversible in a competition assay with fractalkine. In this assay, IBZM is pre-incubated with US28 expressing cells (at concentrations of 0-10 μg/mL) and removed by competition with fractalkine (see FIG. 3).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for diagnosis of CMV, said method comprising administering to a subject having CMV, an image-generating amount of a compound having the formula: 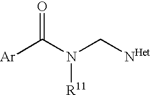
or a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

2. A method in accordance with claim 1, wherein Ar is substituted phenyl.

3. A method in accordance with claim 1, wherein Ar is substituted phenyl and N^{Het} is selected from the group consisting of substituted or unsubstituted pyrrolidinyl, substituted or unsubstituted piperazinyl, substituted or unsubstituted piperidinyl, substituted or unsubstituted morpholinyl and substituted or unsubstituted piperidyl.

4. A method in accordance with claim 1, wherein said compound has the formula: 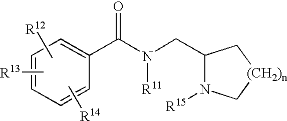
or a pharmaceutically acceptable salt thereof; wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

5. A method in accordance with claim 1, wherein said compound is labeled with a radioisotope selected from the group consisting of ¹⁸F, ⁷⁵Br, ¹²³I and ¹²⁵I.

6. A method in accordance with claim 4, wherein n is 1; R¹¹ is H; R¹², R¹³ and R¹⁴ are each independently selected from the group consisting of H, hydroxy, halogen, (C₁-C₄)alkyl and (C₁-C₄)alkoxy; and R¹⁵ is (C₁-C₄)alkyl.

7. A method in accordance with claim 4, wherein said compound is IBZM.

8. A method in accordance with claim 4, wherein said compound is ¹²³I-IBZM.

9. A method for treating CMV in a human, comprising administering an effective amount of a compound which blocks the binding of a chemokine to US28 or a US28 fragment.

10. A method in accordance with claim 9, wherein said compound has the formula: 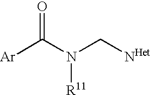
or a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

11. A method in accordance with claim 10, wherein Ar is substituted phenyl.

12. A method in accordance with claim 10, wherein Ar is substituted phenyl and N^{Het} is selected from the group consisting of substituted or unsubstituted pyrrolidinyl, substituted or unsubstituted piperazinyl, substituted or unsubstituted piperidinyl, substituted or unsubstituted morpholinyl and substituted or unsubstituted piperidyl.

13. A method in accordance with claim 10, wherein said compound has the formula: 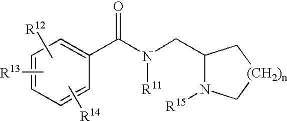
or a pharmaceutically acceptable salt thereof; wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

14. A method in accordance with claim 13, wherein n is 1, R¹¹ is H, R¹⁵ is (C₁-C₄)alkyl, and R¹², R¹³ and R¹⁴ are all other than H.

15. A method in accordance with claim 13, wherein n is 1; R¹¹ is H; R¹², R¹³ and R¹⁴ are each independently selected from the group consisting of H, hydroxy, halogen, (C₁-C₄)alkyl and (C₁-C₄)alkoxy; and R¹⁵ is (C₁-C₄)alkyl.

16. A method for reducing cell motility in a CMV-infected cell, said method comprising contacting said CMV-infected cell with a motility-reducing amount of a compound that inhibits chemokine binding to US28 on the surface of said infected cell.

17. A method in accordance with claim 16, wherein said chemokine is a member selected from the group consisting of fractalkine, MIP-1α, MIP-1β, MCP-1 and RANTES.

18. A method in accordance with claim 16, wherein said chemokine is fractalkine.

19. A method in accordance with claim 16, wherein said compound has the formula: 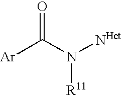
or a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

20. A method in accordance with claim 19, wherein Ar is substituted phenyl.

21. A method in accordance with claim 19, wherein Ar is substituted phenyl, and N^{Het} is selected from the group consisting of substituted or unsubstituted pyrrolidinyl, substituted or unsubstituted piperazinyl, substituted or unsubstituted piperidinyl, substituted or unsubstituted morpholinyl and substituted or unsubstituted piperidyl.

22. A method in accordance with claim 16, wherein said compound has the formula: 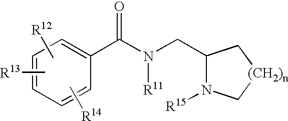
or a pharmaceutically acceptable salt thereof; wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

23. A method in accordance with claim 22, wherein n is 1, R¹¹ is H, R¹⁵ is (C₁-C₄)alkyl, and R¹², R¹³ and R¹⁴ are all other than H.

24. A method in accordance with claim 22, wherein n is 1; R¹¹ is H; R¹², R¹³ and R¹⁴ are each independently selected from the group consisting of H, hydroxy, halogen, (C₁-C₄)alkyl and (C₁-C₄)alkoxy; and R¹⁵ is (C₁-C₄)alkyl.

25. A method in accordance with claim 22, wherein said compound is IBZM or a pharmaceutically acceptable salt thereof.
